SEQUENCE LISTING (19 sequences)

SEQ ID NO: 1 - DNA, 750 bp, Homo sapiens
atgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatatatggacagagacaatggaaaaataaagaagactgttggggtactcaagaactctcttgtgaccttaccagtgaaacctcagacatacaggaaccttattacgggaggaggggcaaaaataaaaataaagggaatccttgggggccaaaacaaagtaaacggaaatcaaaggggaaccagaagaccaacacagtgactgccccagctgccctgaaggcatttgctggatgtgcaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatga

SEQ ID NO: 2 - protein, 249 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRRGKNKNKGNPWGPKQSKRKSKGNQKTNTVTAPAALKAFAGCAKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 3 - DNA, 1255 bp, Homo sapiens
aactcactttaccactactcgctatagagccctggtcaagttctctccacctctctatctatgtctcagtttcttcatctgtaacatcaaatgaataataataccaatctcctagacttcataagaggattaacaaagacaaaatatgggaaaaacataacatggcgtcccataattattagatcttattattgacactaaaatggcattaaaattaccaaaaggaagacagcatctgtttcctctttggtcctgagctggttaaaaggaacactggttgcctgaacagtcacacttgcaaccatgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatgacttgtggaatttggcattcagcaatgtggaaattctaaagctccctgagaacaggatgactcgtgtttgaaggatcttatttaaaattgtttttgtattttcttaaagcaatattcactgttacaccttggggacttctttgtttacccattcttttatcctttatatttcatttgtaaactatatttgaacgacattccccccgaaaaattgaaatgtaaagatgaggcagagaataaagtgttctatgaaatgc

SEQ ID NO: 4 - protein, 231 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 5 - DNA, 810 bp, Homo sapiens
cacttgcaaccatgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagccggggagggcacttactggcaacagcagtgtctattttgtgcagtacaaaatcatgttctcatgcagcatgaaaagctctcaccagaagccaagtggatgctggcagcacatttcttgtaacttcccaggctgcagaacattggctaaatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgccccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcggagaagtgaagagagatgtgtggaaattccatgacttgtgg

SEQ ID NO: 6 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSRRSEERCVEIP

SEQ ID NO: 7 - DNA, 298 bp, Rattus sp.
ttggtgggaaacaaaactagatcctccggtcgtgactataacccgagttaatgcatctttgagggttcgtctccgccccccagagttgccacatagaaaccaaactggaaaaaatacgtccatggaaaattactacaacttagtataccgagtttccataatcaacaattcactggagaaggaacaaaaagcctacgaaggaactcagagagctgttgaaatccaaggtctgacacctcattgcagttactgcgtagtggctgaaatgtaccagcccatgttagacagaagaagtcgg

SEQ ID NO: 8 - DNA, 23 bp, Artificial Sequence
  Description of Artificial Sequence Primer
ctcagtcaacgcatgagtctctg

SEQ ID NO: 9 - DNA, 19 bp, Artificial Sequence
  Description of Artificial Sequence Primer
caggctgccattgcaaaat

SEQ ID NO: 10 - DNA, 28 bp, Artificial Sequence
  Description of Artificial Sequence Probe
agcctcagagggtacaatttcagtcccg

SEQ ID NO: 11 - DNA, 25 bp, Artificial Sequence
  Description of Artificial Sequence Primer
agtctggcttatatccaacacttcg

SEQ ID NO: 12 - DNA, 22 bp, Artificial Sequence
  Description of Artificial Sequence Primer
gactttgctttccttggtcagg

SEQ ID NO: 13 - DNA, 25 bp, Artificial Sequence
  Description of Artificial Sequence Probe
tttcaccagcaagcttcgaccttga

SEQ ID NO: 14 - DNA, 36 bp, Artificial Sequence
  Description of Artificial Sequence Probe
tgcagtacaaaatatatggacagagacaatgaaaaa

SEQ ID NO: 15 - DNA, 21 bp, Artificial Sequence
  Description of Artificial Sequence Primer
aggctgcagaacattggctaa

SEQ ID NO: 16 - DNA, 26 bp, Artificial Sequence
  Description of Artificial Sequence Primer
ttcactggtaaggtcacaagagagtt

SEQ ID NO: 17 - DNA, 36 bp, Artificial Sequence
  Description of Artificial Sequence Probe
tggacagagacaatggaaaaataaagaagactgttg

SEQ ID NO: 18 - DNA, 27 bp, Artificial Sequence
  Description of Artificial Sequence Primer
ctcagtcaacgcatgagtctctgaagc

SEQ ID NO: 19 - DNA, 28 bp, Artificial Sequence
  Description of Artificial Sequence Primer
atagacactgctgttgccagtaagtgcc

(19) United States
(12) Patent Application Publication
(10) Pub. No.: US 2003/0022827 A1
(43) Pub. Date: Jan. 30, 2003

(54) Three new members of the cytokine receptor family class 2

(76) Inventors: Bertram Weiss, Berlin (DE); Robert Sabat, Berlin (DE); Khusru Asadullah, Potsdam (DE); Luisella Toschi, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/961,404

(22) Filed: Sep. 25, 2001

Related U.S. Application Data
(60) Provisional application No. 60/254,896, filed on Dec. 13, 2000.

(30) Foreign Application Priority Data
Sep. 25, 2000 (DE) 100 48 626.6
Nov. 17, 2000 (DE) 100 58 907.3
Dec. 19, 2000 (DE) 100 64 906.8

(51) Int. Cl. A61K 38/17; C07K 14/715; C07H 21/04

(52) U.S. Cl. 514/12; 530/350; 536/23.5

(57) ABSTRACT

This invention relates to three new members of the cytokine receptor class 2 family and their pharmaceutical use.

The cytokine receptor family class 2 was defined based on the structural similarity between proteins that are all highly homologous to fibronectin type III. All previously described members of this family consist of three domains: an extracellular domain, a transmembrane domain and an intracellular domain. The extracellular domain comprises about 200 amino acids and consists of two subdomains with 100 amino acids each. These subdomains contain preserved cysteines, prolines and tryptophans, which play a role in the characteristic folding of the subdomains. The intracellular domain is responsible for the initiation of signal transduction. The first step in this respect is the binding of a ligand (e.g., interleukin-10) to the extracellular domain of receptor 1 (e.g., IL-10R1). The association with receptor 2 (e.g., IL-10R2) produces a complex. In most cases, receptor 2 is not able by itself to bind the ligands, but its association into ligand-receptor complex 1 is essential for directing the signal into the interior of the cell/nucleus.

Cytokine receptor family class 2 (CRF2) comprises receptors for immune mediators such as interleukin 10 (IL-10), interferon γ (IFN-γ), IFN-α and IFN-β, which are of decisive importance for the initiation, course and shut-down of immune reactions. These mediators are already being used in clinical practice.

IL-10, for example, is part of the most important inhibitory mediators of the immune system. It inhibits the specific immune response. In this case, the influence of the antigen-presenting cells seems to be of special importance. IL-10 avoids the development and maturation of dendritic cells from monocytes. Moreover, it reduces the expression of MHC class II and co-stimulatory molecules such as B7-2 of the monocytes and macrophages and thus a suitable antigen presentation of these cells. In addition, IL-10 inhibits the secretion of interleukin-12, a mediator, which plays an important role in the generation of cellular immunity. IL-10 inhibits not only the specific, cellular immune response but also negatively influences the non-specific immune reactions. It inhibits the secretion of pro-inflammatory cytokines such as TNF-α, IL-1β, IL-6, IL-8, GM-CSF of monocytes, macrophages and neutrophilic granulocytes. Moreover, it stimulates the release of antiinflammatory mediators such as IL-1RA and soluble TNF-α receptors. The importance of this cytokine for limiting specific and non-specific immune reactions is confirmed by the phenotype of the IL-10-deficient mice that develop serious inflammations of the intestine, which proceed in a latent manner. This overflowing immune reaction in “normal” food antigens can be overcome by IL-10 administration. After good compatibility was detected in the first administration of IL-10 in healthy test subjects, IL-10 is now being used for therapeutic purposes in diseases such as, e.g., psoriasis, Crohn's disease, and rheumatoid arthritis, which are characterized by a strong, pathological immune activation. IL-10 exerts a positive influence, however, on certain cell populations, such as, e.g., NK-cells, which has a special importance for defense against viral infections and tumors. It stimulates the cytotoxic activity of the NK-cells. Moreover, it increases the IL-2-induced proliferation and cytokine production of these cells.

INF-γ is one of the most potent stimulators of the immune system. It activates the monocytes and macrophages, enhances the respiratory burst and thus the capacity of these cells to kill not only phagocytized microbes but also, under certain conditions, tumor cells. Moreover, IFN-γ stimulates the cytotoxic activity of the NK-cells. In addition, it enhances the specific immune response. It increases the expression of MHC class I and induces the expression of MHC class II in a whole series of cells. Then, it acts directly on T and B cells and promotes their differentiation. As an expression of the deficient activation of monocytes and macrophages, the IFN-γ-deficient mice show high sensitivity to experimental infections with, e.g., mycobacteria, parasites and viruses. IFN-γ is used therapeutically in the reconstruction and the stimulation of the immune system.

Patient studies showed that an increased risk of infections with mycobacteria correlates to a defect in the interferon-γ-receptor 1 (IFN-γR1) (M. J. Newport et al. 1996, N. Engl. J. Med. 335 (26): 1941-9). A point mutation in this receptor results in that the receptor is not expressed on cell surfaces. Nevertheless, Newport et al. found that IFN-γ has an effect on certain functions of the monocytes of these patients who have the mutated receptor. They provide three different hypotheses as an explanation for these seemingly contradictory results. One of these is that there is optionally an additional, previously unidentified receptor for IFN-γ.

For specific influencing of immune reactions, it is therefore necessary to find other previously unknown cytokine receptors.

This invention provides three new members of the cytokine receptor class 2 family.

A subject of this invention is a nucleic acid, which comprises

a. the nucleotide sequence that is shown in Seq ID No 1, Seq ID No 3 or Seq ID No 5,

b. a nucleotide sequence that corresponds to the sequence that consists of a) within the framework of the degeneration of the genetic code or

c. a nucleotide sequence that hybridizes with the sequences of a. and/or b. under stringent conditions, provided that the nucleic acid is different from the genomic sequence and that the nucleic acid codes for a polypeptide with the biological activity of a cytokine receptor.

These nucleic acids code for polypeptides that are new members of the family of the cytokine receptor class 2 or for sections thereof. The cytokine receptor family class 2 is named CRF2 below. These nucleic acids are of human origin. The corresponding sequences also exist in other mammals. They are highly homologous to the sequences according to the invention and can therefore be obtained from corresponding DNA libraries with the aid of the human sequence with the method that is known to one skilled in the art. A comparison between human and rat sequences is shown in FIG. 4.

The term “hybridization under stringent conditions” according to this invention is defined in Sambrook et al. (Molecular Cloning, A Laboratory Manual, Cold Spring Harbor Laboratory Press, 1989). A stringent hybridization exists, for example, if a hybridization signal is observed after washing for one hour with 1×SSC and 0.1% SDS at 50° C., preferably at 55° C., especially preferably at 62° C. and most preferably at 68° C., especially for one hour in 0.2×SSC and 0.1% SDS at 55° C., preferably at 62° C. and most preferably at 68° C. The nucleic acids, which are hybridized under these conditions with the nucleic acid that is shown in Seq ID No 1, Seq ID No 3 or Seq ID No 5 or a nucleotide sequence that corresponds to this sequence within the framework of the degeneration of the genetic code, are also subjects of this invention.

The nucleic acid according to the invention is preferably a DNA. It can also comprise an RNA, however.

The nucleic acid according to the invention can be obtained from a cDNA library or a genomic library produced from human cells

The term genomic sequence is used according to this invention for the genomic sequence that corresponds to the DNA according to the invention, which is found on chromosome 6q24.1-25.2. In public data banks, clone 503F13 is accessible (Accession No. AL050337). It is known that the latter contains the gene for the interferon-(-receptor. It is not known, however, that another gene lies in this chromosome section. This invention identifies the beginning (78552 bp) and the end of the gene (106834 bp) and three different intron/exon structures of the gene. The three intron/exon structures result in three different splice variants of the gene.

In contrast to all previously known members of the CRF2, the three new members of this family according to the invention, with the sequence that is described in Seq ID No 2, Seq ID No 4 and Seq ID No 6, is not membrane-fixed. They have no transmembrane domains, but rather only one extracellular domain. They are secreted by the cells in which they are produced and then are present in bodily fluids as soluble receptors.

The cytokine receptors according to the invention can bind to their specific ligand, the cytokine, but since they are soluble receptors, i.e., are not anchored to a cell membrane, they cannot pass on any signals into the interior of the cell. As a result, the action of the cytokine is limited or eliminated.

There is also the possibility, however, that the cytokine is transported by the binding to the soluble receptor at another site of the organism. It is protected from proteolytic degradation by the binding to a cytokine receptor according to the invention. As a result, the cytokine can exert its biological action long after its release from cells and primarily at another site in the organism. In this case, the cytokine is again released from the complex with the cytokine receptor according to the invention and can bind to membrane-fixed receptors.

In addition, a cytokine receptor according to the invention, after it has bonded to its specific cytokine, can associate with receptor-negative cells and thus trigger certain biological effects. In this case, the production of the complex from a soluble receptor and the cytokine is the requirement for biological effects of the cytokine.

The invention also relates to a nucleic acid that comprises a protein-coding section of the nucleotide sequence that is shown in Seq ID No 1, Seq ID No 3 or Seq ID No 5. The nucleic acid that is shown in Seq ID No 1 contains an open reader frame that extends from position 1 to position 750 and corresponds to a polypeptide with the length of 249 amino acids.

The nucleic acid that is shown in Seq ID No 3 contains an open reader frame that extends from position 304 to position 996 and corresponds to a polypeptide with the length of 231 amino acids.

The nucleic acid that is shown in Seq ID No 5 contains an open reader frame that extends from position 12 to position 800 and corresponds to a polypeptide with the length of 263 amino acids.

Subjects of the invention are also nucleic acids that have a more than 80%, preferably more than 90% and especially preferably more than 95% identity with the nucleotide sequence that is shown in Seq ID No 1, Seq ID No 3 or Seq ID No 5.

In addition, the invention relates to a nucleic acid according to the invention that codes for a cytokine receptor class 2 or a section thereof.

Also subjects of the invention are polypeptides that are coded by a nucleic acid according to the invention. The polypeptides according to the invention preferably have the amino acid sequence that is shown in SEQ ID No 2, Seq ID No 4 or Seq ID No 6, or an identity of more than 80%, preferably more than 90% and especially preferably more than 95% with the amino acid sequence according to SEQ ID No 2, Seq ID No 4 or Seq ID No 6.

The invention also relates to a vector that has at least a copy of a nucleic acid according to the invention or a section thereof. Vectors can be prokaryotic or eukaryotic vectors. Examples of vectors are pPRO (Clontech), pBAD (Invitrogen), pSG5 (Stratagene), pCl (Promega), pIRES (Clontech), pBAC (Clontech), PMET (Invitrogen), pBlueBac (Invitrogen). The nucleic acid according to the invention can be introduced into these vectors with the methods that are known to one skilled in the art. The nucleic acid according to the invention is preferably connected to the vector with expression signals such as, e.g., promoters and enhancers.

In addition, the subject of the invention is a cell that is transformed with a nucleic acid according to the invention or a vector according to the invention. As cells, e.g., E. coli, yeast, Pichia, Sf9, COS, CV-1 or BHK can be used. With commonly used methods, the cell can be transformed with a vector that contains the nucleic acid according to the invention.

The invention also relates to antibodies against a polypeptide according to the invention. An antibody against a polypeptide according to the invention can be monoclonal or polyclonal. It can be directed against the entire polypeptide according to the invention or against fragments thereof. Extraction of such an antibody is done according to standard methods by immunization of test animals.

In addition, the subject of the invention is a pharmaceutical composition, which as an active component contains a nucleic acid according to the invention, a vector according to the invention, a cell according to the invention, an antisense nucleic acid that is directed against the nucleic acid sequence according to Seq ID No 1, Seq ID No 3 or Seq ID No 5, a polypeptide according to the invention or an antibody according to the invention.

The antisense nucleic acid according to the invention is a DNA and/or RNA that is complementary to an mRNA according to the invention. It can comprise the entire complementary sequence or partial sequences.

The pharmaceutical compositions of the invention are produced with commonly used solid or liquid vehicles or diluents and the commonly used pharmaceutical and technical adjuvants corresponding to the desired type of administration with a suitable dosage in a way that is known in the art. Tablets can be obtained, for example, by mixing the active ingredient with known adjuvants, for example inert diluents, such as dextrose, sugar, sorbitol, mannitol, polyvinylpyrrolidone, explosives such as corn starch or alginic acid, binders such as starch or gelatins, lubricants such as carboxypolymethylene, carboxymethylcellulose, cellulose acetate phthalate or polyvinyl acetate. Capsules that contain active ingredients can be produced, for example, by the active ingredient being mixed with an inert vehicle such as lactose or sorbitol and encapsulated in gelatin capsules.

The pharmaceutical compositions according to the invention can also be used in suitable solutions, such as, for example, physiological common salt solution.

For parenteral administration, especially oily solutions, such as, for example, solutions in sesame oil, castor oil, and cottonseed oil, are suitable. To increase the solubility, solubilizers, such as, for example, benzylbenzoate or benzyl alcohol, can be added.

The subject of the invention is also the use of a pharmaceutical composition according to the invention for diagnosis in immune diseases or in reproduction medicine.

The term immune diseases is used herein for immune diseases in the more limited sense (e.g., autoimmune diseases) and for diseases on whose course the immune system has a decisive impact, such as, e.g., tumor diseases and chronic/life-threatening infections (insufficient immunological response).

Under physiological conditions, i.e., in the healthy human, the cytokine receptors according to the invention are very strongly expressed in the placenta and in the mammary gland. This uncommon tissue-specific expression makes possible the use of pharmaceutical compositions according to the invention for diagnosis, prevention and treatment of diseases of the reproductive and immune systems.

Placenta and mammary glands play an important role immunologically in the reproduction and development of offspring. The immunological balance between mother and child is controlled almost exclusively by these two organs. The placenta ensures that the fetus, which immunologically must actually be identified as foreign, is not rejected like, e.g., a transplanted organ, but rather tolerated. This phenomenon of immunological tolerance is achieved by immunomodulating factors that are produced in the placenta. At the same time, the placenta is also co-responsible for the “normal”, development of the immune system of the fetus. Immediately after birth, the infant must successfully deal with his environment with the immunological dangers. Since at this time, however, he still does not have a completely efficient immune system, he is supported by the so-called loaned immunity from the mother. In this case, breast milk plays a decisive role. Mediators that influence the immune system of the infant are found in breast milk.

The cytokine receptors according to the invention are factors in these immunomodulatory systems of the placenta and the mammary gland.

Under pathophysiological conditions, the cytokine receptor according to the invention is also expressed in tissues in which only a slight expression can be detected in the healthy state. A considerable difference in the expression can be measured in, e.g., the skin. In healthy skin, the expression is slight, but in the case of psoriasis, an inflammatory skin disease with immunological background, it increases considerably. The third variant (Seq ID No 5) is expressed especially in the skin of patients with atopic dermatitis and cutaneous T-cell-lymphoma.

Psoriasis is an important model disease, which has essential similarities to other immune diseases, in which a type 1 cytokine pattern is of decisive importance. These include, i.a., rheumatoid arthritis, multiple sclerosis, inflammatory intestinal diseases such as Crohn's disease and ulcerative colitis, as well as the transplant rejections after organ transplants. Psoriasis offers the possibility of generating exemplary knowledge for these diseases with a similar pathophysiological mechanism (Asadullah et al., Drugs of Today 1999, 35 (12): 913-924; Romagnani, S. J. Clin Immunol 1995, 15:121-9).

This varying expression in healthy and diseased tissue makes possible the use of the pharmaceutical composition according to the invention for diagnosis, prevention and treatment of immune diseases, such as psoriasis, rheumatoid arthritis, multiple sclerosis, inflammatory intestinal diseases such as Crohn's disease and ulcerative colitis, as well as transplant rejections after organ transplants.

Immune diseases frequently accompany an altered expression or a mutation of a cytokine receptor. This invention makes possible the diagnosis of cytokine receptors according to the invention in immune diseases, especially in reproduction medicine. For example, an improper immune reaction can result in the fetus being rejected. The diagnosis can be carried out on the basis of the nucleic acid or the polypeptide. To this end, either blood or tissue samples are taken from patients. In these samples, the amount of polypeptide according to the invention can be determined by an immune test. In this regard, antibodies are produced from a polypeptide according to the invention or from fragments thereof, and the latter are then used in an ELISA (enzyme-linked-immunosorbent assay), in an RIA (radioimmunoassay) or in immunohistochemistry. As an alternative, RNA from the samples is purified, and the amount of mRNA according to the invention is measured by Northern Blot, PCR or chip-hybridization. In the chip-hybridization, nucleic acids according to the invention or fragments thereof are present on special nucleic acid chips. The amount of nucleic acid according to the invention can also be determined by in situ hybridization with antisense-RNA, which is directed against a nucleic acid according to the invention. In this case, the antisense-RNA can be labeled with digoxigenin, ³²P or ³³P.

With the aid of the above-described diagnosis, mutations can also be detected. Thus, e.g., by hybridization of the patient-DNA or -RNA with a nucleic acid sequence according to the invention, mutations of individual nucleotides can be detected.

The results of the diagnosis can then be correlated with the clinical picture of the patient.

The pharmaceutical composition according to the invention can be used for the production of a medication for treating or preventing diseases of the reproductive and immune systems that are based on a relative deficiency of one or more cytokine receptors according to the invention. This can be done by the administration of recombinant protein or the use of gene therapy. In this case, a vector that contains a nucleic acid according to the invention is designed and administered. Examples are vectors that are derived from adenovirus, adenovirus-associated virus, Herpes simplex virus or SV40. The gene therapy can be implemented according to a protocol as described by Gomez-Navarro et al. (Eur. J. Cancer (1999) 35, 867-885). The administration can be done locally, i.e., directly in the tissue in question or systemically, i.e., via the circulation. This produces an increased expression of the polypeptide according to the invention.

The pharmaceutical composition according to the invention can also be used for treatment or prevention of diseases of the reproductive and immune systems, which are based on a relative over-expression of one or more cytokine receptors according to the invention. In this regard, an antisense nucleic acid can be used, which prevents the expression of the nucleic acid according to the invention. In addition, antibodies according to the invention can be used that inhibit the biological activity of the receptor.

The use of the pharmaceutical composition according to the invention for the production of a medication for treatment or prevention of psoriasis is preferred.

In addition, the pharmaceutical composition according to the invention can be used for influencing the binding of a ligand to other membrane-fixed receptors. A cytokine receptor according to the invention can bind, e.g., a ligand of the receptor and thus influence the action of the ligand on the cell. Immune diseases that can be attributed to excessive concentration of the ligand thus can be treated.

A polypeptide according to the invention or a nucleic acid according to the invention can be used for tracing ligands of the cytokine receptors according to the invention. In addition to known cytokines, previously unknown cytokines can also bind as ligands to the receptor. The measurement can be done with various methods. One option is to bind the polypeptide according to Seq ID No 2, Seq ID No 4 or Seq ID No 6 or portions thereof to a solid matrix, to bring it into contact with solutions to be tested and to identify the bonded substances. Solutions to be tested can be, for example, bodily fluids or a mixture of peptides or polypeptides that are produced in a synthetic or recombinant manner. These peptides or polypeptides can be labeled with, e.g., biotin.

In addition, a polypeptide according to the invention or a nucleic acid according to the invention can be used for tracing modulators of cytokine receptors according to the invention. These modulators modulate the binding of the natural ligand to the receptor. They can act both as antagonists and as agonists. Antagonists inhibit the binding of the natural ligand to the receptor and can be used to treat diseases that are based on an excessive activity of the cytokine. Agonists enhance the binding of the natural ligand to the receptor and can be used in patients who have a reduced concentration of the natural ligand or a slight expression of the receptor. Modulators can be identified in a binding assay.

The invention is explained in more detail by the following figures and examples.

### FIGURES

FIG. 1 shows the tissue expression of the cytokine receptors according to the invention. The expression is plotted as a multiple of the expression of HPRT.

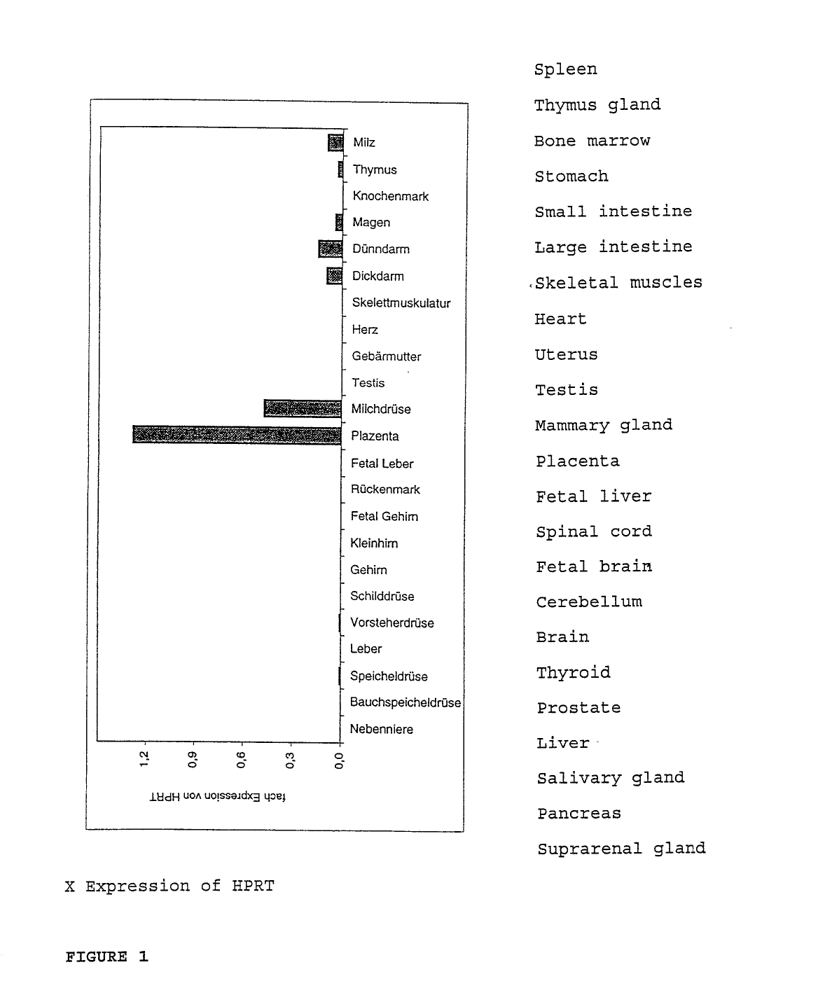

FIG. 2 shows a comparison of the amino acids of Seq No 2 (1st line in each case), Seq ID No 4 (2nd line in each case) and Seq ID No 6 (3rd line in each case) and the identification of the sequences in terms of exons.

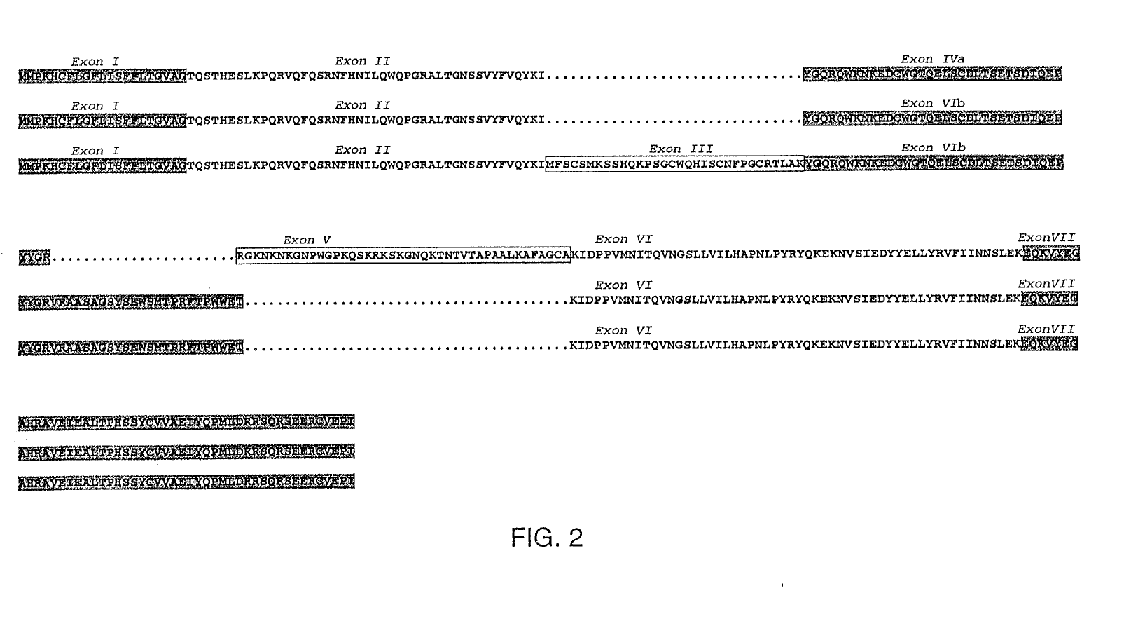

FIG. 3 shows the expression of the cytokine receptor according to the invention with the sequence that is described in Seq ID No 5 in the skin of patients with cutaneous T-cell-lymphoma (CTCL) and atopic dermatitis (AD) in comparison to the skin of healthy test subjects.

FIG. 4 shows a comparison between a human cytokine receptor (Seq ID No 3) and the corresponding rat sequence. The upper sequence in each case is the human sequence, and the lower sequence is the rat sequence.

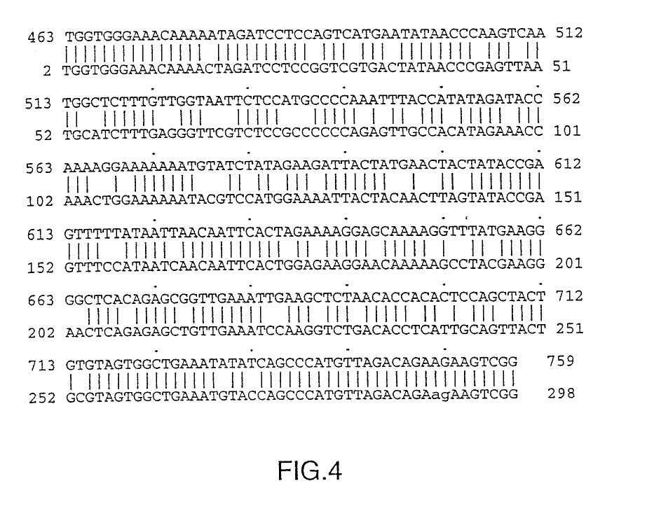

FIG. 5 shows a partial sequence of the cytokine receptor of rats.

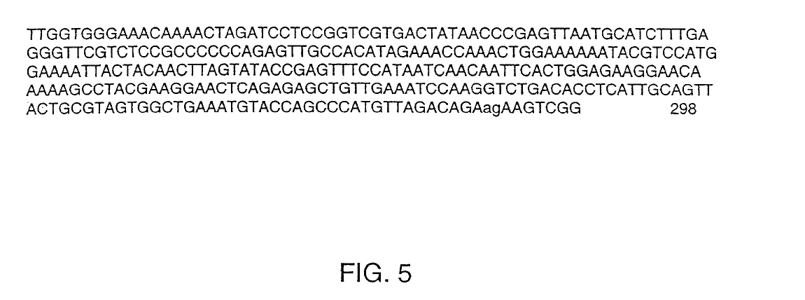

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosures of all applications, patents and publications, cited above, and of corresponding German Application No. 100 48 626.6, filed Sep. 25, 2000; German Application No. 100 58 907.3, filed Nov. 17, 2000 and German Application No. 100 64 906.8, filed Dec. 19, 2000 as well as U.S. Provisional Application Serial No. 60/243,286, filed Oct. 26, 2000 and Provisional Application Serial No. 60/254,896, filed Dec. 13, 2000 are hereby incorporated by reference.

### EXAMPLES

### Example 1

### Detection of a New Cytokine Receptor Based on the Varying Action of Human IL-10 and EVB-coded Protein BCRF1

Four viral IL-10 homologs, from the Ebstein-Barr virus, from the cytomegalovirus, from equine Herpes virus type 2 and from the site-virus, are described. The Ebstein-Barr virus codes a protein BCRF1, whose amino acid sequence is 83% identical to the human IL-10 (hIL-10). Most differences are based on the deletion of the N-terminal area of the human cytokine and as a result have a weaker binding of the BCRF1 to IL-10R1. BCRF1 plays an important role in the interaction between the virus and the infected organism, i.e., when an EBV infection spreads. We studied the biological action of BCRF1 on human monocytes, which are in vivo the main target cells of the human cytokine. We characterized the influence of both mediators on the LPS-induced release of TNF-α and on the expression of various surface molecules of the monocytes (MHC class II, co-stimulatory molecules, receptors for immunoglobulins). The lymphocytes and the monocytes (PBMC) were separated by a density-gradient centrifuging from the anticoagulated venous blood of the healthy donor. To determine the inhibitory influence of hIL-10 and BCRF1 on the LPS-induced TNF-A release, these cells were pre-incubated for one hour with various concentrations of BCRF1 or hIL-10 at 37° C. and in the moist 5% CO₂-containing atmosphere. Then, the cells were stimulated with 100 ng/ml of LPS for 3 hours in the same surrounding area. Reduction of the cell-free culture supernatant and the determination of the TNF-α using ELISA resulted. Human IL-10 inhibits the TNF-α release in a dose-dependent manner. BCRF1 exerts a similar action that is probably expressed, however, only at higher concentrations (starting from about 3 ng/ml) based on the weaker binding to IL-10R1 of the viral homolog. We also characterize the influence of the two mediators on the expression of various surface molecules of the human monocytes. The separated PBMC were incubated for 24 hours at various concentrations of BCRF1 or hIL-10 at 37° C. and in the moist 5% CO₂-containing atmosphere. Then, the expression of the surface molecules of the monocytes was determined using continuous-flow cytometry. Human IL-10 simultaneously inhibits in a dose-dependent manner the expression of MHC class II but enhances the expression of the receptors for the immunoglobulin class G (CD16, CD32, CD64). BCRF1 again induces similar changes. Advantageously at high concentrations of BCRF1 (30 ng/ml to 100 ng/ml), a maximum enhancement of the expression of the low-affine IgG receptor CD16 that is identical to hIL-10 is induced. Equal concentrations of BCRF1 are not able to exert a maximum action on the expression of the high-affine IgG-receptor CD64, which is identical to the expression induced by hII-10, however. This variable adjustment, in one case identical to that of hIL-10 (CD16) and in the other case different (CD64), demonstrates the existence of two IL-10R subtypes. Subtype I, which consists of an IL-10R1 and an IL-10R2, is responsible for the ramping up of the expression of CD16. Both hIL-10 and BCRF1 bind to it. Instead of II-10R1, subtype-2 contains a new member of the cytokine receptor family class 2. hIL-10R1 but not BCRF1 binds to subtype 2. Both subtypes are responsible for the adjustment of the expression of CD64. Since BCRF1 binds to subtype 1, it exerts a specific influence on the expression, but since BCRF1 is not able to bind the subtype-2, it cannot exert the maximum hIL-10-identical action.

### Example 2

### Expression in Various Human Tissues

The expression of cytokine receptors according to the invention in various human tissues was determined by means of a quantitative RT-PCR method. To this end, a “real time PCR” (TaqMan-PCR) was used.

The elimination of some contaminating genomic DNA and the reverse transcription of mRNA were performed as follows: 2 μg of total RNA was incubated at 37° C. for 30 minutes in 40 μl with 5× moloney murine leukemia virus of reverse transcriptase (M-MLV RT) buffer (Gibco BRL, Eggenstein, Germany) and 10 mmol/L of dithiothreitol, 250 μmol/L respectively of DATP, dUTP, dGTP and dCTP (Pharmacia Biotech, Uppsala, Sweden), 1 unit/μL of RNasin ribonuclease inhibitor (Promega, Mannheim, Germany), 25 ng/μL of random hexadeoxynucleotide primer (Promega) and 0.05 unit/μL of RQ1 DNase (Promega). The samples were then heated to 95° C. for 10 minutes and then cooled with ice. After 1 unit/μL of RNasin ribonuclease inhibitor (Promega) and 5 units/μL of M-MLV RT (Gibco BRL) were added, the samples were incubated for 10 minutes at room temperature and subsequently for one hour at 37° C. Then, the enzymes were inactivated by heating to 95° C. for 10 minutes. The resulting cDNA was analyzed in each case in 3 batches (3-fold determination) by means of the real-time TaqMan PCR with use of an ABI Prism 7700 sequence detector system (Perkin-Elmer Cetus, Forster City, Calif., U.S.A.). For the detection of the cDNA sequence of all three variants of the cytokine receptor according to the invention, the following PCR primer and a fluorochromium-labeled oligonucleotide probe were designed with use of the Primer Express software (Perkin-Elmer Cetus):

| | |
|---|---|
| Sense | CTCAGTCAACGCATGAGTCTCTG |
| primer: | |
| Antisense | CAGGCTGCCATTGCAAAAT |
| primer: | |
| Probe: | FAM-AGCCTCAGAGGGTACAATTTCAGTCCCG-TAMRA. |

FAM stands for 6-carboxy-fluorescein, TAMRA stands for 6-carboxy-tetramethyl-rhodamine.

Moreover, the mRNA content of the homo sapiens hypoxanthine phosphoribosyltransferase-1 (HPRT-1) that codes for the “house-keeping gene” and is regarded as an internal control was determined in parallel in each individual sample. The sequence of the HPRT-1-specific primer pair and the fluorogenic probe, based on the reverse complimentary cDNA strand, was as follows:

| | |
|---|---|
| AGTCTGGCTTATATCCAACACTTCG | (sense |
| | primer), |
| GACTTTGCTTTCCTTGGTCAGG | (antisense |
| | primer), |
| FAM-TTTCACCAGCAAGCTTCGACCTTGA-TAMRA | (sense |
| | probe). |

The PCR amplification was performed with use of 1 μL of reverse transcription mix in a final volume of 50 μL, which contained the TaqMan universal master mix (Applied Biosystems, Weiterstadt, Germany) and 200 nmol/L of the specific probe and 900 (CRF2-n3) or 300 (HPRT-1) nmol/L of the respective primer. The above-mentioned concentrations had previously been identified as optimal in preliminary tests. Significant fluorescence signals were defined as the threshold value cycle that was calculated using the TaqMan instrument-associated software version 1.6.3 (Applied Biosystems). The cytokine receptor-mRNA content was calculated in relation to that for HPRT-1.

The tissue-specific analysis of the gene expression showed that the cytokine receptors according to the invention are expressed in particular in the placenta and in the mammary gland (here even stronger than the housekeeping genes).

### Example 3

### Expression under Pathophysiological Conditions

The study should answer the questions of whether the expression of the cytokine receptors according to the invention can be regulated and whether it is distinguishable between physiological and pathophysiological states. To this end, RNA from skin biopsies from healthy test subjects was used with RNA from skin biopsies from patients with psoriasis, atopic dermatitis (AD) and cutaneous T-cell-lymphoma (CTCL). The determination was performed as described in Example 2.

The determination produced an over-10-times-stronger expression of the cytokine receptors according to the invention in the lesional skin of patients with psoriasis in comparison to the skin of healthy test subjects.

In another test, probes were used that can differ between the second variant (Seq ID No 3) and the third variant (Seq ID No 5):

Variant 2 (Seq ID No 3):

| | |
|---|---|
| Sense primer: | CTCAGTCAACGCATGAGTCTCTG |
| Antisense primer: | CAGGCTGCCATTGCAAAAT |
| Probe: | FAM-5′-TGCAGTACAAAATATATGGACAGAGACAATGGAAAAA-3′-TAMRA. |
| Variant 3 | (Seq ID No 5): |

| | |
|---|---|
| Sense primer: | 5′-AGGCTGCAGAACATTGGCTAA-3′ |
| Antisense primer: | 5′-TTCACTGGTAAGGTCACAAGAGAGTT-3′ |
| Probe: | FAM-5′-TGGACAGAGACAATGGAAAAATAAAGAAGACTGTTG-3′-TAMPA |

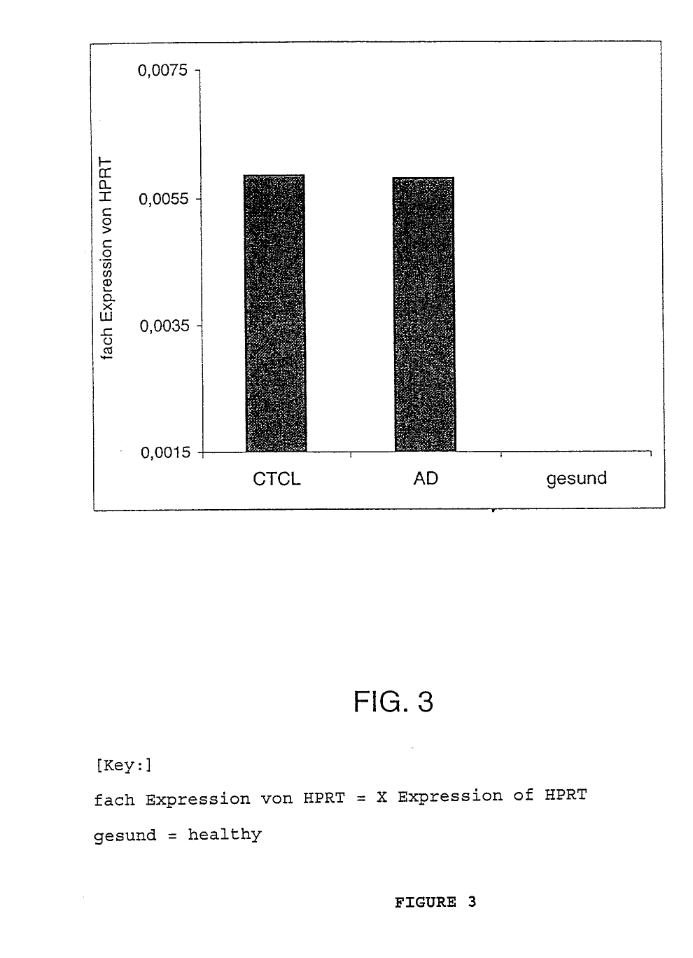

The expression of the third variant (Seq ID No 5) is significantly increased in comparison to healthy skin in the case of CTCL and AD (see FIG. 3).

### Example 4

### Cloning of Cytokine Receptors According to Seq ID No 1 and No 3

First, a cDNA library was produced from human placenta. To this end, 4 μg of total RNA (Clontech Company) was used in the reactions of the GeneRacer™ kit (Invitrogen, Calif., USA). The modified mRNA from the last reaction was used with the GeneRacer™ OligodT primer for reverse transcription.

The cDNA for the cytokine receptor was obtained by means of a RACE-PCR (RACE=Rapid Amplification of cDNA Ends). The DNA-polymerase for the PCR was the ThermoZyme™ from Invitrogen.

| | Batch Volumes |
|---|---|
| | 50 μl |
| 5′-RACE | |
| Placenta cDNA | 2 μl |
| GeneRacer ™ 5′ primer (10 μM) | 1 μl |
| Gene-specific primer (GSP) #2R (25 μM) | 1 μl |
| 5x ThermoZyme ™ PCR buffer | 10 μl |
| dNTP solution (10 mM each) | 1 μl |
| ThermoZyme ™ (1 U/μl) | 1 μl |
| Sterile water | 34 μl |
| 3′-RACE | |
| Placenta cDNA | 2 μl |
| GeneRacer ™ 3′ primer (10 μM) | 1 μl |
| GSP #1F (35 μM) | 1 μl |
| 5x ThermoZyme ™ PCR buffer | 10 μl |
| dNTP solution (10 mM each) | 1 μl |
| ThermoZyme ™ (1 U/μl) | 1 μl |
| Sterile water | 34 μl |

The following “cycling” programs were used for the RACE-PCR in the thermocycler Perkin-Elmer 9600:

| | | |
|---|---|---|
| 94° C. | 2 min | 1 cycle |
| 94° C. | 30 sec | 5 cycles |
| 72° C. | 3 min | |
| 94° C. | 30 sec | 5 cycles |
| 70° C. | 30 sec | |
| 72° C. | 3 min | |
| 94° C. | 30 sec | 25 cycles |
| 68° C. | 30 sec | |
| 72° C. | 3 min | |
| 72° C. | 10 min | 1 cycle |

The sequences of the cytokine receptor primer for the RACE reactions were as follows:

| | |
|---|---|
| #1F | |
| 5′-CTC AGT CAA CGC ATG ACT CTC TGA AGC-3′ | (sense primer) |
| #2R | |
| 5′-ATA GAC ACT GCT GTT GCC AGT AAG TGC C-3′ | (antisense primer) |

Then, the products of RACE in the pCR^{(R)}4-TOPO vector were cloned from the TOPO-TA cloning^{(R)} kit from Invitrogen.

The sequence of the cloned POR products was determined by means of the Applied Biosystem (ABI) Prism^{(R)} BigDye™ terminator sequencing kit in an ABI 310 capillary sequencer.

### Example 6

### Cloning of the Cytokine Receptor According to Seq ID No 5

The cytokine receptor was identified from human placenta and mammary glands of the cDNA library. The placenta of the cDNA library was produced by means of the GeneRacer™ kit (Invitrogen, Calif., USA). The mammary gland of the cDNA library was used by the Clontech Company (Marathon-Ready™ cDNA).

The cDNA for the cytokine receptor was obtained by means of a PCR amplification (RT-PCR).

The DNA-polymerase for the PCR was the Advantage^{(R)} 2 of CLONTECH.

The RT-PCR was performed as follows:

| | Batch Volumes |
|---|---|
| RT-PCR | 50 μl |
| Placenta/mammary gland cDNA | 2 μl |
| CRF2-n4 #1a-F primer (20 μm) | 1 μl |
| CRF2-n4 #2b-R primer (20 μm) | 1 μl |
| 10x cDNA PCR reaction buffer | 5 μl |
| dNTP mix (10 mmol) | 1 μl |
| 50x Advantage ® 2 polymerase mix | 1 μl |
| Sterile water | 36 μl |

The following “cycling” programs were used for the RT-PCR in the thermocycler Perkin-Elmer 9600:

| | | |
|---|---|---|
| 94° C. | 30 sec | 1 cycle |
| 94° C. | 5 sec | 30 cycles |
| 68° C. | 3 min | |

The sequences of the CRF2-n4 primer for the RT-PCR were as follows:

| | |
|---|---|
| #1a-F | |
| 5′-CAC TTG CAA CCA TGA TGC CTA AAC A-3′ | (sense |
| | primer) |
| #2b-R | |
| 5′-CCA CAA GTC ATG GAA TTT CCA CAC A-3′ | (antisense |
| | primer) |

Then, the products of RT-PCR in the pCR^{(R)}4-TOPO vector were cloned from the TOPO-TA cloning^{(R)} kit from Invitrogen.

The sequence of the cloned PCR products was determined by means of the Applied Biosystem (ABI) Prism^{(R)} BigDye™ terminator sequencing kit in an ABI 310 capillary sequencer.

### Example 6

### Expression of the Cytokine Receptors

To obtain the purified recombinant proteins, the corresponding cDNAs was cloned in an E. coli expression vector.

The cytokine receptors were expressed as fusion proteins with the glutathione-S-transferase (GST) in the vector pGEX-2T (Pharmacia Biotech). The fusion construct was then transformed into the E. coli strain BL21.

### Example 7

### Search for Modulators

The cytokine receptors according to the invention are expressed in E. coli and purified with the aid of standard methods, such as, e.g., ion exchange chromatography, affinity chromatography or gel filtration. The purified protein is bonded to microtiter plates. Then, the biotin-labeled ligand and the substance to be tested are added simultaneously and incubated for 1 hour. Then, the microtiter plate is washed with buffer, and then avidin-FITC is added. After another 30 minutes of incubation, it is washed again, and then the fluorescence is measured. In control batches, only the labeled ligand is added. By measuring the fluorescence, it can be determined whether the substance that is to be tested displaces the ligand from the receptor (fluorescence is lower), has no effect (fluorescence corresponds to that of the control batch) or enhances the binding of the ligand (fluorescence becomes stronger). Analogous tests are performed with ¹²⁵I-labeled ligands.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Nucleic acid, characterized in that it comprises
a. the nucleotide sequence that is shown in Seq ID No 1, Seq ID No 3 or Seq ID No 5,
b. a nucleotide sequence that corresponds to the sequence that consists of a) within the framework of the degeneration of the genetic code or
c. a nucleotide sequence that hybridizes with the sequences of a. and/or b. under stringent conditions, and that codes for a polypeptide with the biological activity of a cytokine receptor,
provided that the nucleic acid is different from the genomic sequence.

2. Nucleic acid according to claim 1, wherein it comprises a protein-coding section of the nucleotide sequence that is shown in SEQ ID No 1, Seq ID No 3 or Seq ID No 5.

3. Nucleic acid according to claim 1 or 2, wherein it has a more than 80Q identity with the nucleotide sequence that is shown in Seq ID No 1, Seq ID No 3 or Seq ID No 5.

4. Nucleic acid according to one of claims 1-3, wherein it codes for a cytokine receptor class 2 or a section thereof.

5. Polypeptide that is coded by a nucleic acid according to one of claims 1-4.

6. Polypeptide according to claim 5, wherein it has the amino acid sequence that is shown in SEQ ID No 2, Seq ID No 4 or Seq ID No 6.

7. Vector, wherein it has at least a copy of a nucleic acid according to one of claims 1-4 or a section thereof.

8. Cell, wherein it is transformed with a nucleic acid according to one of claims 1-4 or a vector according to claim 8.

9. Antibody against a polypeptide according to one of claims 5-7.

10. Pharmaceutical composition, wherein as active components, it contains
a. a nucleic acid according to one of claims 1-4,
b. a vector according to claim 7,
c. a cell according to claim 8,
d. an antisense nucleic acid that is directed against the nucleic acid sequence according to Seq ID No 1, Seq ID No 3 or Seq ID No 5,
e. a polypeptide according to one of claims 5-6, or
f. an antibody according to claim 9.

11. Use of a pharmaceutical composition according to claim 10 for diagnosis in immune diseases or in reproduction medicine.

12. Use of a pharmaceutical composition according to claim 11 for the production of a medication for treatment and prevention of diseases of the reproductive or immune systems.

13. Use according to claim 12, whereby the immune disease is psoriasis.

14. Use of a polypeptide according to one of claims 5-6 or a nucleic acid according to one of claims 1-4 for tracing ligands of a polypeptide according to claim 5.

15. Use of a polypeptide according to one of claims 5-6 or a nucleic acid according to one of claims 1-4 for tracing modulators of a polypeptide according to claim 5.
